(19) United States
(12) Patent Application Publication
(10) Pub. No.: US 2026/0166122 A1
(43) Pub. Date: Jun. 18, 2026

(54) USE OF MICROPEPTIDE MIAC

(71) Applicant: NANJING ANJI BIOTECHNOLOGY CO., LTD, Nanjing (CN)

(72) Inventors: Hanmei XU, Nanjing (CN); Setrerrahmane SARRA, Nanjing (CN)

(21) Appl. No.: 19/389,942

(22) Filed: Nov. 14, 2025

Related U.S. Application Data
(63) Continuation of application No. PCT/CN2024/093260, filed on May 15, 2024.

(30) Foreign Application Priority Data
May 15, 2023 (CN) 202310544241.1

(51) Int. Cl. A61K 38/17; A61P 35/00; C12Q 1/6886

(52) U.S. Cl. CPC A61K 38/1709; A61P 35/00; C12Q 1/6886; C12Q 2600/158

(57) ABSTRACT

Use of a micropeptide MIAC, which belongs to the technical field of biomedicine. The present disclosure specifically relates to use of the micropeptide MIAC in preparation of a reagent or a medicament for detecting, preventing or treating tumors. The tumors comprise one or more of solid tumors and hematologic malignancies, such as pancreatic cancer, hepatocellular carcinoma, colorectal cancer, ovarian cancer, cervical cancer, bladder cancer, melanoma, glioblastoma, neuroblastoma, glioma, osteosarcoma, lymphoma, hematologic malignancies, myeloma, cholangiocarcinoma and prostate cancer. The micropeptide MIAC has the effects of inhibiting growth, proliferation and/or migration of various tumor cells, has a wide treatment spectrum, and is suitable for diagnosing, preventing or treating various tumors, specifically malignant tumors.

### SEQUENCE LISTING

This application contains a Sequence Listing which has been submitted electronically in WIPO Standard ST.26 (XML format) and is hereby incorporated by reference in its entirety. Said Sequence Listing copy, created on Nov. 11, 2025, is named “G204RAYT0009US.xml” and is 4,824 bytes in size.

### TECHNICAL FIELD

The present application belongs to the technical field of biomedicine, and specifically relates to use of a micropeptide MIAC.

### BACKGROUND

An open reading frame (ORF) is a potential translatable sequence consisting of a series of in-frame sense codons, and these codons start with an initiation codon and end with a termination codon. Translatable ORFs are typically labeled as coding DNA sequences (CDSs) on an mRNA, and these sequences are responsible for protein translation.

Long noncoding RNAs (lncRNAs) are a type of noncoding RNAs longer than 200 nt, expressed in a tissue- and developmental stage-specific manner, and have been shown to directly regulate multiple functions. lncRNAs also contain short open-reading frames (sORFs), which are generally less than 300 nt in length and were previously thought to lack a coding ability. However, recent studies have shown that some sORFs have the coding ability, and their encoded products are defined as micropeptides.

More and more micropeptides with fewer than 150 amino acids have been discovered in various organisms, from bacteria to humans. The functional diversity of these micropeptides and the emergence of new methods for identifying them have attracted the attention of the scientific community. All these studies indicate that micropeptides translated from sORFs play important regulatory roles in many key physiological processes, such as muscle development, amino acid metabolism, immune regulation, tumor growth, and cell death.

Maolei Zhang et al. identified an lncRNA with a potential coding ability through circular RNA sequencing (circRNAseq) and ribosome nascent chain complex sequencing (RNC-seq) analysis of glioma cells. Experiments revealed that its second exon could form a circular RNA (circRNA), which was translated into a micropeptide of 87 amino acids. In vitro and in vivo experiments demonstrated that this micropeptide could inhibit the proliferative capacity of glioma and interact with a polymerase complex-associated factor (PAF1) to suppress the transcriptional elongation of multiple proto-oncogenes. Clinical sample analysis showed that compared with para-carcinoma tissues, this micropeptide exhibits significantly down-regulated expression in breast cancer, hepatocellular carcinoma, and gastric cancer, suggesting its potential role in the diagnosis, treatment and the like related to malignant tumors (Zhang M, et al. A peptide encoded by circular form of LINC-PINT suppresses oncogenic transcriptional elongation in glioblastoma. Nat Commun. 2018 Oct. 26; 9 (1): 4475.).

In related technologies, for example, the Chinese invention patent with publication number CN112442116A describes use of a micropeptide encoded by an lncRNA in the detection and treatment of malignant tumors such as head and neck cancer, thyroid cancer, and renal carcinoma.

However, different tumor tissues exhibit genetic, phenotypic, and immune heterogeneity, and research on micropeptides, particularly in tumor treatment, remains limited, so that it is necessary to explore their uses in the treatment of different tumors. In order to further expand the spectrum of other tumors where MIAC may play a role, the present disclosure attempts to detect the expression of MIAC in various tumor cells and corresponding normal cells, and systematically evaluates the activity of the micropeptide MIAC in different tumors through in vitro and in vivo experiments, so as to provide a new solution for tumor treatment.

### SUMMARY

### 1. Purpose of the Invention

The purpose of the present application is to provide use of a micropeptide MIAC. After long-term exploration and continuous attempts, the inventors have found that the micropeptide MIAC having an amino acid sequence as shown in SEQ ID NO: 1 can be used in preparation of a reagent or a medicament for detecting, preventing or treating tumors such as pancreatic cancer, hepatocellular carcinoma, colorectal cancer, ovarian cancer, cervical cancer, bladder cancer, melanoma, glioblastoma, neuroblastoma, glioma, osteosarcoma, lymphoma, hematologic malignancies, myeloma, cholangiocarcinoma and prostate cancer.

### 2. Technical Solutions

To address the above technical problem, the technical solutions adopted in the present application are as follows:

The present application provides use of a micropeptide MIAC in preparation of a reagent or a medicament for detecting, preventing or treating tumors, the micropeptide MIAC has an amino acid sequence as shown in SEQ ID NO: 1, and the amino acid sequence of SEQ ID NO: 1 is: MERAGVPGFSPRRSSVEAKMQSTSCSVRKSSTVTAWPAVVLLLSWGQRRGG.

Further, the tumors comprise one or more of pancreatic cancer, hepatocellular carcinoma, colorectal cancer, ovarian cancer, cervical cancer, bladder cancer, melanoma, glioblastoma, neuroblastoma, glioma, osteosarcoma, lymphoma, hematologic malignancies, myeloma, cholangiocarcinoma and prostate cancer.

Further, the reagent or the medicament for preventing or treating tumors comprises at least the micropeptide MIAC having the amino acid sequence as shown in SEQ ID NO: 1 and a pharmaceutically acceptable carrier thereof.

Further, the preventing or treating tumors comprises inhibiting growth, migration and/or proliferation of tumor cells.

Further, the preventing or treating tumors comprises inhibiting the proliferation of tumor cells.

Further, the preventing or treating tumors comprises inhibiting the migration of tumor cells. Further, the preventing or treating tumors comprises inhibiting the growth of tumor cells, especially the growth of tumor cells in vivo.

Further, the tumor cells in the inhibiting the proliferation of tumor cells include:
cancer cells of the pancreatic cancer, comprising: any one or a combination of MIP-PaCa-2, Aspc-1 and PANC-1;
cancer cells of the hepatocellular carcinoma, comprising: any one or a combination of HepG2, SMMC-7721, HCCLM3, QGY-7701, and Hep3B;
cancer cells of the colorectal cancer, comprising: any one or a combination of LOVO, SW480, and HCT-116;
cancer cells of the ovarian cancer, comprising: SKOV3 and/or A2780;
cancer cells of the cervical cancer, comprising: Hela and/or SIHA;
cancer cells of the bladder cancer, comprising: T24 and/or EJ;
cancer cells of the melanoma, comprising: any one or a combination of A375, SK-mel-2, and M14;
cancer cells of the glioblastoma, comprising: U87-MG;
cancer cells of the neuroblastoma, comprising: SH-SY5Y;
cancer cells of the glioma, comprising: SHG-44;
cancer cells of the osteosarcoma, comprising: any one or a combination of MG-63, Saos-2, and U-20S;
cancer cells of the lymphoma, comprising: any one or a combination of U937, Raji, U2932, WSU-DLCL2, OCI-LY10, and JeKo-1;
cancer cells of the hematologic malignancies, comprising: any one or a combination of NB4, Reh, K562, Thp-1, JurKat, and HL60;
cancer cells of the myeloma, comprising: any one or a combination of NCI-H929, ARP-1 and RPMI-8226;
cancer cells of the cholangiocarcinoma, comprising: QBC939;
and/or,
cancer cells of the prostate cancer, comprising: Du145 cells.

Further, the tumor cells in the inhibiting the migration of tumor cells comprise: prostate cancer Du145 cells, cervical cancer Hela cells, osteosarcoma MG-63 cells, ovarian cancer SKOV3 cells, melanoma A375 cells, and/or hepatocellular carcinoma Hep3B cells.

Further, the tumor cells in the inhibiting the growth of tumor cells comprise: hepatocellular carcinoma Hep3B cells.

The present application also provides use of a nucleotide encoding an amino acid sequence as shown in SEQ ID NO: 1 in preparation of a reagent or a medicament for detecting, preventing or treating tumors.

Further, the tumors comprise one or more of pancreatic cancer, hepatocellular carcinoma, colorectal cancer, ovarian cancer, cervical cancer, bladder cancer, melanoma, glioblastoma, neuroblastoma, glioma, osteosarcoma, lymphoma, hematologic malignancies, myeloma, cholangiocarcinoma and prostate cancer.

Further, the nucleotide comprises a nucleotide sequence as shown in SEQ ID NO: 2, and the nucleotide sequence as shown in SEQ ID NO: 2 is:

atggagagagcaggggtgcccgggttctctccgcggcgctcatcgg
tggaggcgaagatgcagagcaccagctgcagtgtcaggaagagctc
cacagtcaccgcctggccagccgtcgtgctgttgctgagctgggga
cagagaagaggcggatga.

The present application further provides use of a recombinant vector in preparation of a reagent or a medicament for detecting, preventing or treating tumors, and the recombinant vector encodes an amino acid sequence as shown in SEQ ID NO: 1 or comprises a nucleotide sequence as shown in SEQ ID NO: 2.

Further, the tumors comprise one or more of pancreatic cancer, hepatocellular carcinoma, colorectal cancer, ovarian cancer, cervical cancer, bladder cancer, melanoma, glioblastoma, neuroblastoma, glioma, osteosarcoma, lymphoma, hematologic malignancies, myeloma, cholangiocarcinoma and prostate cancer.

A pharmaceutical composition for treating tumors is provided, and the pharmaceutical composition comprises at least the micropeptide MIAC, the nucleotide, or the recombinant vector, and a pharmaceutically acceptable carrier thereof; and the tumors comprise one or more of pancreatic cancer, hepatocellular carcinoma, colorectal cancer, ovarian cancer, cervical cancer, bladder cancer, melanoma, glioblastoma, neuroblastoma, glioma, osteosarcoma, lymphoma, hematologic malignancies, myeloma, cholangiocarcinoma and prostate cancer.

The present application also provides a method for detecting, preventing or treating tumors, and the method comprises administering the micropeptide MIAC having the amino acid sequence as shown in SEQ ID NO: 1 or a pharmaceutical composition comprising the micropeptide MIAC to a subject with such need.

Further, the subject may be a human, a mouse, or a monkey.

Further, in the method, the micropeptide MIAC is administered to the subject at a dose of 0.1-50 mg/kg, 0.1-20 mg/kg, 0.1-10 mg/kg, 0.1-5 mg/kg, 5-20 mg/kg, 10-20 mg/kg, or 5-15 mg/kg.

### 3. Beneficial Effects

Compared with the related art, the present application has the following beneficial effects.

(1) The expression level of the micropeptide MIAC provided in the present application in human pancreatic cancer, hepatocellular carcinoma, colorectal cancer, ovarian cancer, cervical cancer, bladder cancer, melanoma, glioblastoma, neuroblastoma, glioma, osteosarcoma, lymphoma, hematologic malignancies, myeloma, cholangiocarcinoma and prostate cancer cells is significantly lower than that in the corresponding normal cells.

(2) Regarding the use of micropeptide MIAC provided in the present application, the micropeptide can significantly inhibit the proliferation of tumor cells in pancreatic cancer, hepatocellular carcinoma, colorectal cancer, ovarian cancer, cervical cancer, bladder cancer, melanoma, glioblastoma, neuroblastoma, glioma, osteosarcoma, lymphoma, hematologic malignancies, myeloma, and cholangiocarcinoma, as well as inhibit the migration of tumor cells in prostate cancer, cervical cancer, osteosarcoma, ovarian cancer, melanoma, and hepatocellular carcinoma.

(3) Regarding the use of micropeptide MIAC provided in the present application, the micropeptide can significantly inhibit the in vivo growth of hepatocellular carcinoma, especially tumors derived from hepatocellular carcinoma Hep3B cells.

(4) The micropeptide MIAC provided in the present application has the effects of inhibiting the proliferation and migration of various tumor cells, as well as inhibiting the in vivo tumor growth of hepatocellular carcinoma. It has a wide treatment spectrum, and is suitable for diagnosing, preventing or treating various tumors, specifically malignant tumors.

### BRIEF DESCRIPTION OF THE DRAWINGS

FIG. 1 shows the expression analysis results of a micropeptide MIAC in different tumor cells and normal cells;

FIG. 2A shows the statistical result of the inhibitory effect of the micropeptide MIAC on the migration activity of prostate cancer Du145 cells;

FIG. 2B shows the cell staining images of a blank group and all administration groups in an experiment of inhibiting the migration activity of prostate cancer Du145 cells by the micropeptide MIAC;

FIG. 3A shows the statistical result of the inhibitory effect of the micropeptide MIAC on the migration activity of cervical cancer Hela cells;

FIG. 3B shows the cell staining images of a blank group and all administration groups in an experiment of inhibiting the migration activity of cervical cancer Hela cells by the micropeptide MIAC;

FIG. 4A shows the statistical result of the inhibitory effect of the micropeptide MIAC on the migration activity of osteosarcoma MG-63 cells;

FIG. 4B shows the cell staining images of a blank group and all administration groups in an experiment of inhibiting the migration activity of osteosarcoma MG-63 cells by the micropeptide MIAC;

FIG. 5A shows the statistical result of the inhibitory effect of the micropeptide MIAC on the migration activity of ovarian cancer SKOV3 cells;

FIG. 5B shows the cell staining images of a blank group and all administration groups in an experiment of inhibiting the migration activity of ovarian cancer SKOV3 cells by the micropeptide MIAC;

FIG. 6A shows the statistical result of the inhibitory effect of the micropeptide MIAC on the migration activity of malignant melanoma A375 cells;

FIG. 6B shows the cell staining images of a blank group and all administration groups in an experiment of inhibiting the migration activity of malignant melanoma A375 cells by the micropeptide MIAC;

FIG. 7A shows the statistical result of the inhibitory effect of the micropeptide MIAC on the migration activity of hepatocellular carcinoma Hep3B cells;

FIG. 7B shows the cell staining images of a blank group and all administration groups in an experiment of inhibiting the migration activity of hepatocellular carcinoma Hep3B cells by the micropeptide MIAC;

FIG. 8 is an in vivo growth curve of a subcutaneously transplanted tumor of hepatocellular carcinoma Hep3B cells in a nude mouse inhibited by the MIAC in Example 4; and

FIG. 9 is a statistical graph of tumor weight on the 22^{nd} day after the MIAC inhibiting the subcutaneously transplanted tumor of hepatocellular carcinoma Hep3B cells in the nude mice in Example 4.

### DETAILED DESCRIPTION

The present application will be further described below in conjunction with specific examples.

It should be noted that terms such as “upper”, “lower”, “left”, “right”, and “middle” cited in the specification are only for the sake of clarity in description and are not intended to limit the scope of implementation. Any changes or adjustments to their relative relationships, without substantial changes to the technical content, shall also be deemed to be within the scope of implementation of the present application.

Unless otherwise defined, all technical and scientific terms used herein have the same meaning as that commonly understood by a person skilled in the art to which the present application belongs to; and the term “and/or” as used herein includes any and all combinations of one or more related listed items.

Where specific conditions are not specified in the examples, experiments shall be carried out according to the conventional conditions or the conditions suggested by the manufacturers. For reagents or instruments whose manufacturers are not indicated, they are all conventional products available for purchase in the market.

As used herein, the term “about” is used to provide flexibility and imprecision associated with a given term, metric, or value. Those skilled in the art can easily determine the degree of flexibility for specific variables.

As used herein, the term “at least one of . . . ” is intended to be synonymous with “one or more of . . . ”. For example, “at least one of A, B and C” explicitly includes only A, only B, only C, and their respective combinations.

Concentrations, amounts, and other numerical data may be presented in a range format herein. It should be understood that such a range format is used merely for convenience and brevity, and should be flexibly interpreted to include not only the numerical values explicitly recited as the limits of the range, but also all individual numerical values or sub-ranges encompassed within the stated range, as if each numerical value and sub-range were explicitly recited. For example, a numerical range of about 1 to about 4.5 should be interpreted to include not only the explicitly recited limits of 1 to about 4.5, but also individual numbers (such as 2, 3, and 4) and sub-ranges (such as 1 to 3, and 2 to 4). The same principle applies to ranges reciting only one numerical value, such as “less than about 4.5”, which should be interpreted to include all of the above values and ranges. Moreover, this interpretation should apply regardless of the breadth of the range or feature being described.

A micropeptide inhibiting actin cytoskeleton (MIAC) is a human endogenous protein or polypeptide fragment with 51 amino acids encoded by a long noncoding RNA (lncRNA). The micropeptide MIAC has an amino acid sequence:

(SEQ ID NO: 1)
MERAGVPGFSPRRSSVEAKMQSTSCSVRKSSTVTAWPAVVLLLSWG
QRRGG.

The present application provides use of a micropeptide MIAC in preparation of a reagent or a medicament for detecting, preventing or treating tumor diseases.

In some implementations, the tumors comprise solid tumors and hematologic malignancies.

In some implementations, the tumors comprise pancreatic cancer, hepatocellular carcinoma, colorectal cancer, ovarian cancer, cervical cancer, bladder cancer, melanoma, glioblastoma, neuroblastoma, glioma, osteosarcoma, lymphoma, hematologic malignancies, myeloma, cholangiocarcinoma and/or prostate cancer.

The micropeptide MIAC provided in the present application has the effect of inhibiting growth, migration and/or proliferation of tumor cells in vivo and/or in vitro.

In some implementations, the micropeptide MIAC has an inhibitory effect on the proliferation of the following various tumor cells:
cancer cells of pancreatic cancer, comprising: any one or a combination of MIP-PaCa-2, Aspc-1 and PANC-1;
cancer cells of hepatocellular carcinoma, comprising: any one or a combination of HepG2, SMMC-7721, HCCLM3, QGY-7701, and Hep3B;
cancer cells of colorectal cancer, comprising: any one or a combination of LOVO, SW480, and HCT-116;
cancer cells of ovarian cancer, comprising: SKOV3 and/or A2780;
cancer cells of cervical cancer, comprising: Hela and/or SIHA;
cancer cells of bladder cancer, comprising: T24 and/or EJ;
cancer cells of melanoma, comprising: any one or a combination of A375, SK-mel-2, and M14;
cancer cells of glioblastoma, comprising: U87-MG;
cancer cells of neuroblastoma, comprising: SH-SY5Y;
cancer cells of glioma, comprising: SHG-44;
cancer cells of osteosarcoma, comprising: any one or a combination of MG-63, Saos-2, and U-2OS;
cancer cells of lymphoma, comprising: any one or a combination of U937, Raji, U2932, WSU-DLCL2, OCI-LY10, and JeKo-1;
cancer cells of hematologic malignancies, comprising: any one or a combination of NB4, Reh, K562, Thp-1, JurKat, and HL60;
cancer cells of myeloma, comprising: any one or a combination of NCI-H929, ARP-1 and RPMI-8226;
cancer cells of cholangiocarcinoma, comprising: QBC939; and
cancer cells of prostate cancer, comprising: Du145 cells.

The micropeptide MIAC provided in the present application has the effect of inhibiting the proliferation of tumor cells within the range of 50-200 μM, 10-200 μM, or 50-100 μM.

In some implementations, the micropeptide MIAC can also inhibit the migration of tumor cells, specifically comprising: human prostate cancer Du145 cells, human cervical cancer Hela cells, human osteosarcoma MG-63 cells, human ovarian cancer SKOV3 cells, human malignant melanoma A375 cells, and/or human hepatocellular carcinoma Hep3B cells.

Optionally, the micropeptide MIAC can inhibit the migration of tumor cells within the range of 0.78-50 μM, 0.78-25 M, 1.56-50 μM, 3.125-50 μM, 6.25-50 μM, 25-50 μM, or 12.5-50 μM.

The micropeptide MIAC provided in the present application can inhibit the in vivo growth of tumor cells. Optionally, the MIAC can inhibit the in vivo tumor growth of human hepatocellular carcinoma cells. In some implementations, the micropeptide MIAC inhibits the in vivo tumor growth of hepatocellular carcinoma Hep3B cells.

Therefore, the present application also provides a method for detecting, preventing or treating tumor diseases, which comprises administering the micropeptide MIAC comprising or having an amino acid sequence as shown in SEQ ID NO: 1 or a pharmaceutical composition comprising the micropeptide MIAC to a subject with such need.

The subject may optionally be a human, a mouse, a monkey, a dog, or a pig. In some implementations, the subject is a human, a monkey, a dog, or a mouse.

When the pharmaceutical composition comprising the micropeptide MIAC is administered to the subject, the pharmaceutical composition further comprises at least one pharmaceutically acceptable excipient.

The term “pharmaceutically acceptable carrier” refers to an excipient or a carrier that does not cause significant irritation to the subject and does not eliminate the biological activity and characteristics of the administered anti-mesothelin antibody or its antigen-binding fragment and/or any additional therapeutic agent in the composition. A pharmaceutically acceptable carrier may enhance or stabilize the composition, or may be used for facilitating the preparation of the composition.

The pharmaceutical composition comprising the micropeptide MIAC may be administered via an appropriate route based on the specific applicable form, physicochemical properties, and the like of the pharmaceutically acceptable carrier. In some implementations, the pharmaceutical composition may be formulated as a lyophilized preparation or a liquid preparation, which may comprise appropriate formulation additives used in the art. For example, the above pharmaceutical composition typically comprises 1 or more pharmaceutical excipients, for example, sterilized liquids such as water and oils (including oils of petroleum, animal, vegetable, or synthetic origin (e.g., peanut oil, soybean oil, mineral oil, and sesame oil)). In the case of intravenous administration of the above pharmaceutical composition, water is a more representative carrier. In addition, saline solutions as well as aqueous glucose solutions and aqueous glycerol solutions may also be used as liquid carriers, particularly for solutions for injection. Appropriate pharmaceutical excipients are known in the art. If necessary, the composition may also comprise trace amounts of wetting or emulsifying agents, or a pH buffering agent. The pharmaceutical composition is generally administered via a parenteral route, which may be intradermal, intramuscular, intraperitoneal, intravenous, or subcutaneous injection, but is not limited thereto; and for example, it may be administered via infusion or bolus injection, or via an intracranial, intrathecal, or intranasal (e.g., inhalation), intradermal, subcutaneous, or transmucosal route.

When the micropeptide MIAC is used for detecting, preventing, or treating tumors, the dosage varies based on factors such as the specific condition being treated, the severity of the condition, individual patient parameters (including age, physical condition, size, gender, and weight), duration of treatment, nature of concurrent therapy (if any), specific route of administration, and the knowledge of the physician or veterinarian. The dosage is gradually increased until the desired effect is achieved. In some implementations, the dosage of an active ingredient can be determined empirically in individuals who have received one or more micropeptide MIAC administrations.

In some implementations, for example, when 0.1-50 mg/kg, 0.1-20 mg/kg, 0.1-10 mg/kg, 0.1-5 mg/kg, 5-20 mg/kg, 10-20 mg/kg or 5-15 mg/kg of the micropeptide MIAC is administered to a subject, the tumorigenicity of tumor cells can be significantly inhibited, and in particular, the in vivo tumorigenicity of tumor cells can be inhibited.

In some implementations, the dose administered to a subject can be converted between species, such as dose conversion between humans and mice. The conversion method is known in the art. See, for example, Experimental Methodology of Pharmacology, Xu Shuyun. Experimental Methodology of Pharmacology (2^{nd} Edition) [M]. People's Medical Publishing House, 1991, the respective contents of which are hereby incorporated by reference.

The technical solutions of the present disclosure will be described more clearly and explicitly below in the following examples in an illustrative manner. It should be understood that these examples are for illustrative purposes only and are by no means intended to limit the scope of protection of the present disclosure. The scope of protection of the present disclosure is limited only by the claims.

### Example 1

This example detects the expression of a micropeptide MIAC in different tumor cells and normal cells.

### (1) Cell Culture and RNA Extraction

Human pancreatic ductal epithelial cells HPDE and pancreatic cancer cells PANC-1, Aspc-1, human normal hepatocytes L02 and hepatocellular carcinoma cells HepG2, QGY-7701, Hep3B, human normal colon cells CCD-18Co and colorectal cancer cells SW480, HCT-116, human normal ovarian epithelial cells IOSE-80 and ovarian cancer cells SKOV3, A2780, human cervical epithelial cells HCerEpiC and cervical cancer cells Hela, SIHA, human normal bladder epithelial cells SV-HUC-1 and bladder cancer cells T24, EJ, human epidermal keratinocytes HEKa and melanoma cells SK-mel-2, M14, human brain glial cells T98G, glioma cells SHG-44, and neuroblastoma cells SH-SY5Y, human osteoblast cell line hFOB1.19 and osteosarcoma cells MG-63, U-2OS, human T lymphocytes H9 and lymphoma cells U2932, WSU-DLCL2, JeKo-1, human peripheral blood mononuclear cells PBMC and hematologic malignancies cells K562, HL60, NCI-H929, RPMI-8226, human intrahepatic bile duct epithelial cells HIBEpiC and cholangiocarcinoma cells QBC939, as well as human prostate epithelial cells HPEpiC and prostate cancer cells Du145 were cultured in an incubator at 37° C. with 5% CO₂ until the density reached 90%, the supernatant was discarded, a certain volume of Trizol (Life Invitrogen) was added, and the total RNA was extracted from different cells according to the instructions.

### (2) Primer Design

Primers were designed based on the information of nucleotide sequences encoding a micropeptide MIAC, with the sequences as follows:

forward primer (SEQ ID NO. 3):
ATGGAGAGAGCAGGGGTGCCCG;
reverse primer (SEQ ID NO. 4):
CGCCTCTTCTCTGTCCCCAGCTC.

### (3) Real-Time Quantitative PCR was Used for Detecting the Expression of the Micropeptide MIAC in Various Tumor Cells and Normal Cells.

The total RNA extracted in the above steps was quantified with a NanoDrop ND-1000 nucleic acid quantifier for the purity and concentration of the extracted RNA, and agarose quality control was performed to ensure the integrity of the extracted RNA. The extracted total RNA was reverse-transcribed to synthesize cDNA using a kit with a product name of PrimeScript™ RT reagent Kit with gDNA Eraser (Perfect Real Time) from TaKaRa. A qPCR reaction was carried out using a kit with a product name of SYBR® Premix Ex Taq™ II (Tli RNaseH Plus) from TaKaRa. The reaction system is shown in Table 1. After the above components were mixed evenly, the following procedures were followed: pre-denaturation at 95° C. for 30 s, followed by 40 cycles; and 95° C. for 5 s, and 60° C. for 30 s.

TABLE 1
PCR reaction system

| Reagent | Usage volume (μL) |
|---|---|
| SYBR Premix Ex Taq II (Tli RNaseH Plus) (2×) | 12.5 |
| PCR Forward Primer (10 μM) | 1 |
| PCR Reverse Primer (10 μM) | 1 |
| DNA template (<100 ng) | 2 |
| Sterilized water | 8.5 |
| Total | 25 |

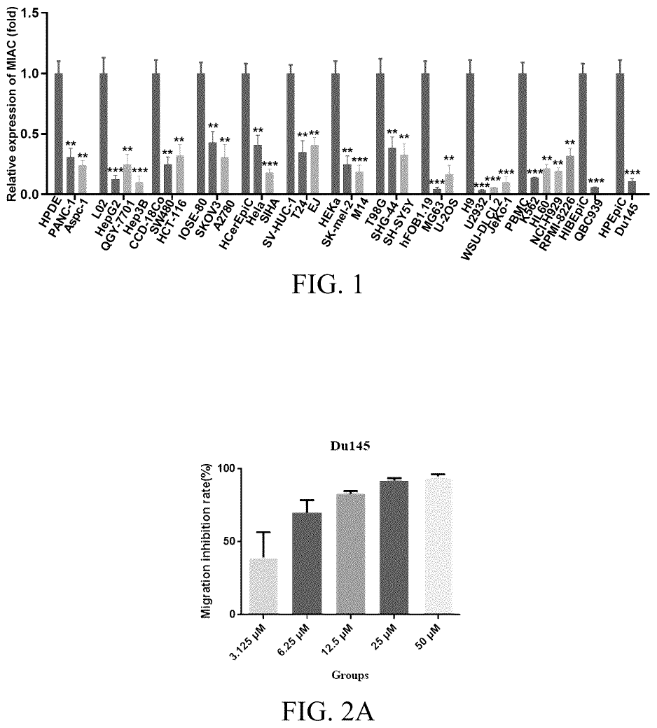

The specificity of the reaction was determined based on a melting curve, and the relative expression of the MIAC was calculated by a formula 2-ΔΔCt. The results are shown in FIG. 1. Compared with the corresponding normal cells, the expression level of the MIAC in human pancreatic cancer, hepatocellular carcinoma, colorectal cancer, ovarian cancer, cervical cancer, bladder cancer, melanoma, glioblastoma, neuroblastoma, glioma, osteosarcoma, myeloma, lymphoma, hematologic malignancies, and cholangiocarcinoma cells was significantly lower than that in normal cells. Therefore, the anti-tumor effect of the MIAC was investigated in the above tumor cells in the future.

### Example 2

This example provides the inhibitory effect of the micropeptide MIAC on the proliferation of human tumor cells.

Human pancreatic cancer, hepatocellular carcinoma, colorectal cancer, ovarian cancer, cervical cancer, bladder cancer, melanoma, glioblastoma, neuroblastoma, glioma, osteosarcoma, myeloma, lymphoma, hematologic malignancies, and cholangiocarcinoma cells were cultured in an incubator at 37° C. with 5% CO₂ until the density reached 90%, then digested and collected with trypsin, resuspended in a culture medium, and counted under a microscope; the cell concentration was adjusted to about 1.0×10⁵ cells/mL; and the cell suspension was inoculated into a 96-well plate, with 100 μL per well, and incubated overnight in an incubator at 37° C. with 5% CO₂. After the cells were completely attached to the wall, 100 μL of micropeptides MIAC with concentrations of 50 μM, 100 μM and 200 μM were added to form an administration group; and a culture medium without any medicament was used as a negative control group. After continuing incubation for 72 h, 100 μL of CCK8 was added to each well of the 96-well plate for dissolving. The absorbance OD was measured at a detection wavelength of 450 nm with a microplate reader, and the proliferation inhibition (PI) was calculated using the formula of PI (%)=1-administration group/negative control group. The experiment was repeated 3 times independently, the results were expressed as Mean±SD, and a statistical T test was performed, where *P<0.05 indicated a significant difference and **P<0.01 indicated an extremely significant difference. The results are shown in Table 2-Table 52.

TABLE 2
Inhibitory effect of micropeptide MIAC on the proliferation
of human pancreatic cancer cells PANC-1

| | | 72 h | |
|---|---|---|---|
| | | | Proliferation |
| Group | | OD | inhibition (%) |
| Blank well | | 0.291 ± 0.002 | — |
| Negative control group | | 1.048 ± 0.041 | — |
| Administration group | 200 μM | 0.427 ± 0.02** | 59.288 ± 1.909** |
| | 100 μM | 0.708 ± 0.043** | 32.443 ± 4.063** |
| | 50 μM | 1.027 ± 0.073 | 2.036 ± 6.961 |

Note:
Compared with the negative control group,
*P < 0.05,
**P < 0.01.

TABLE 3
Inhibitory effect of micropeptide MIAC on the proliferation
of human pancreatic cancer cells Aspc-1

| | | 72 h | |
|---|---|---|---|
| | | | Proliferation |
| Group | | OD | inhibition (%) |
| Blank well | | 0.291 ± 0.002 | — |
| Negative control group | | 0.948 ± 0.003 | — |
| Administration group | 200 μM | 0.509 ± 0.019** | 46.273 ± 2.035** |
| | 100 μM | 0.711 ± 0.047** | 24.965 ± 4.971** |
| | 50 μM | 0.939 ± 0.015 | 0.949 ± 1.575 |

Note:
Compared with the negative control group,
*P < 0.05,
**P < 0.01.

TABLE 4
Inhibitory effect of micropeptide MIAC on the proliferation
of human pancreatic cancer cells MIP-PaCa-2

| | | 72 h | |
|---|---|---|---|
| | | | Proliferation |
| Group | | OD | inhibition (%) |
| Blank well | | 0.257 ± 0.003 | — |
| Negative control group | | 1.033 ± 0.014 | — |
| Administration group | 200 μM | 0.571 ± 0.038** | 44.739 ± 3.716** |
| | 100 μM | 0.866 ± 0.035** | 16.107 ± 3.372** |
| | 50 μM | 0.898 ± 0.035 | 13.041 ± 3.422 |

Note:
Compared with the negative control group,
*P < 0.05,
**P < 0.01.

TABLE 5
Inhibitory effect of micropeptide MIAC on the proliferation
of human esophageal carcinoma cells Eca-109

| | | 72 h | |
|---|---|---|---|
| | | | Proliferation |
| Group | | OD | inhibition (%) |
| Blank well | | 0.291 ± 0.003 | — |
| Negative control group | | 1.064 ± 0.023 | — |
| Administration group | 200 μM | 0.394 ± 0.039** | 63.001 ± 3.636** |
| | 100 μM | 0.656 ± 0.045** | 38.315 ± 4.205** |
| | 50 μM | 1.072 ± 0.037 | −0.752 ± 3.493 |

Note:
Compared with the negative control group,
*P < 0.05,
**P < 0.01.

TABLE 6
Inhibitory effect of micropeptide MIAC on the proliferation
of human esophageal carcinoma cells CaES-17

| | | 72 h | |
|---|---|---|---|
| | | | Proliferation |
| Group | | OD | inhibition (%) |
| Blank well | | 0.312 ± 0.003 | — |
| Negative control group | | 1.217 ± 0.030 | — |
| Administration group | 200 μM | 0.466 ± 0.022** | 61.692 ± 1.819** |
| | 100 μM | 0.672 ± 0.051** | 44.770 ± 4.194** |
| | 50 μM | 1.168 ± 0.044 | 4.080 ± 3.650 |

Note:
Compared with the negative control group,
*P < 0.05,
**P < 0.01.

TABLE 7
Inhibitory effect of micropeptide MIAC on the proliferation
of human gastric carcinoma cells BGC-803

| | | 72 h | |
|---|---|---|---|
| | | | Proliferation |
| Group | | OD | inhibition (%) |
| Blank well | | 0.247 ± 0.004 | — |
| Negative control group | | 1.484 ± 0.009 | — |
| Administration group | 200 μM | 0.715 ± 0.015** | 51.853 ± 1.022** |
| | 100 μM | 0.960 ± 0.059** | 35.347 ± 3.963** |
| | 50 μM | 1.379 ± 0.025 | 7.074 ± 1.656 |

Note:
Compared with the negative control group,
*P < 0.05,
**P < 0.01.

TABLE 8
Inhibitory effect of micropeptide MIAC on the proliferation
of human gastric carcinoma cells BGC-823

| | | 72 h | |
|---|---|---|---|
| | | | Proliferation |
| Group | | OD | inhibition (%) |
| Blank well | | 0.291 ± 0.002 | — |
| Negative control group | | 1.128 ± 0.019 | — |
| Administration group | 200 μM | 0.327 ± 0.025** | 71.040 ± 2.217** |
| | 100 μM | 0.556 ± 0.029** | 50.680 ± 2.588** |
| | 50 μM | 0.950 ± 0.045** | 15.780 ± 3.992** |

Note:
Compared with the negative control group,
*P < 0.05,
**P < 0.01.

TABLE 9
Inhibitory effect of micropeptide MIAC on the proliferation
of human hepatocellular carcinoma cells HepG2

| | | 72 h | |
|---|---|---|---|
| | | | Proliferation |
| Group | | OD | inhibition( %) |
| Blank well | | 0.192 ± 0.003 | — |
| Negative control group | | 0.832 ± 0.006 | — |
| Administration group | 200 μM | 0.169 ± 0.015** | 79.696 ± 1.770** |
| | 100 μM | 0.267 ± 0.022** | 67.962 ± 2.687** |
| | 50 μM | 0.575 ± 0.047** | 30.877 ± 5.587** |

Note:
Compared with the negative control group,
*P < 0.05,
**P < 0.01.

TABLE 10
Inhibitory effect of micropeptide MIAC on the proliferation
of human hepatocellular carcinoma cells SMMC-7721

| | | 72 h | |
|---|---|---|---|
| | | | Proliferation |
| Group | | OD | inhibition (%) |
| Blank well | | 0.215 ± 0.002 | — |
| Negative control group | | 1.407 ± 0.004 | — |
| Administration group | 200 μM | 0.674 ± 0.033** | 52.132 ± 2.309** |
| | 100 μM | 1.032 ± 0.011** | 26.670 ± 0.749** |
| | 50 μM | 1.292 ± 0.012 | 8.171 ± 0.860 |

Note:
Compared with the negative control group,
*P < 0.05,
**P < 0.01.

TABLE 11
Inhibitory effect of micropeptide MIAC on the proliferation
of human hepatocellular carcinoma cells HCCLM3

| | | 72 h | |
|---|---|---|---|
| | | | Proliferation |
| Group | | OD | inhibition (%) |
| Blank well | | 0.234 ± 0.002 | — |
| Negative control group | | 1.470 ± 0.025 | — |
| Administration group | 200 μM | 0.706 ± 0.041** | 51.962 ± 2.795** |
| | 100 μM | 0.981 ± 0.019** | 33.273 ± 1.308** |
| | 50 μM | 1.420 ± 0.054 | 3.402 ± 3.691 |

Note:
Compared with the negative control group,
*P < 0.05,
**P < 0.01.

TABLE 12
Inhibitory effect of micropeptide MIAC on the proliferation
of human hepatocellular carcinoma cells QGY-7701

| | | 72 h | |
|---|---|---|---|
| | | | Proliferation |
| Group | | OD | inhibition (%) |
| Blank well | | 0.215 ± 0.002 | — |
| Negative control group | | 1.398 ± 0.009 | — |
| Administration group | 200 μM | 0.520 ± 0.019** | 62.837 ± 1.374** |
| | 100 μM | 0.785 ± 0.030** | 43.862 ± 2.111** |
| | 50 μM | 1.185 ± 0.018** | 15.232 ± 1.260** |

Note:
Compared with the negative control group,
*P < 0.05,
**P < 0.01.

TABLE 13
Inhibitory effect of micropeptide MIAC on the
proliferation of human colon cancer cells LOVO

| | | 72 h | |
|---|---|---|---|
| | | | Proliferation |
| Group | | OD | inhibition (%) |
| Blank well | | 0.247 ± 0.004 | — |
| Negative control group | | 1.339 ± 0.014 | — |
| Administration group | 200 μM | 0.624 ± 0.026** | 53.373 ± 1.965** |
| | 100 μM | 0.970 ± 0.021** | 27.558 ± 1.574** |
| | 50 μM | 1.148 ± 0.008** | 14.289 ± 0.608** |

Note:
Compared with the negative control group,
*P < 0.05,
**P < 0.01.

TABLE 14
Inhibitory effect of micropeptide MIAC on the
proliferation of human colon cancer cells SW480

| | | 72 h | |
|---|---|---|---|
| | | | Proliferation |
| Group | | OD | inhibition (%) |
| Blank well | | 0.192 ± 0.003 | — |
| Negative control group | | 0.812 ± 0.024 | — |
| Administration group | 200 μM | 0.147 ± 0.016** | 81.904 ± 2.015** |
| | 100 μM | 0.283 ± 0.007** | 65.121 ± 0.801** |
| | 50 μM | 0.640 ± 0.032** | 21.215 ± 3.968** |

Note:
Compared with the negative control group,
*P < 0.05,
**P < 0.01.

TABLE 15
Inhibitory effect of micropeptide MIAC on the proliferation
of human colon cancer cells HCT-116

| | | 72 h | |
|---|---|---|---|
| | | | Proliferation |
| Group | | OD | inhibition (%) |
| Blank well | | 0.247 ± 0.004 | — |
| Negative control group | | 1.441 ± 0.013 | — |
| Administration group | 200 μM | 0.688 ± 0.047** | 52.255 ± 3.273** |
| | 100 μM | 0.976 ± 0.038** | 32.269 ± 2.653** |
| | 50 μM | 1.253 ± 0.019** | 13.046 ± 1.340** |

Note:
Compared with the negative control group,
*P < 0.05,
**P < 0.01.

TABLE 16
Inhibitory effect of micropeptide MIAC on the
proliferation of human breast cancer cells MX-1

| | | 72 h | |
|---|---|---|---|
| | | | Proliferation |
| Group | | OD | inhibition (%) |
| Blank well | | 0.257 ± 0.003 | — |
| Negative control group | | 1.069 ± 0.008 | — |
| Administration group | 200 μM | 0.630 ± 0.051** | 41.098 ± 4.761** |
| | 100 μM | 0.808 ± 0.102** | 24.415 ± 9.554** |
| | 50 μM | 1.020 ± 0.028 | 4.584 ± 2.574 |

Note:
Compared with the negative control group,
*P < 0.05,
**P < 0.01.

TABLE 17
Inhibitory effect of micropeptide MIAC on the proliferation
of human breast cancer cells MDA-MB-231

| | | 72 h | |
|---|---|---|---|
| | | | Proliferation |
| Group | | OD | inhibition (%) |
| Blank well | | 0.198 ± 0.003 | — |
| Negative control group | | 1.120 ± 0.009 | — |
| Administration group | 200 μM | 0.206 ± 0.016** | 81.602 ± 1.437** |
| | 100 μM | 0.378 ± 0.058** | 66.270 ± 5.216** |
| | 50 μM | 0.777 ± 0.050** | 30.604 ± 4.508** |

Note:
Compared with the negative control group,
*P < 0.05,
**P < 0.01.

TABLE 18
Inhibitory effect of micropeptide MIAC on the proliferation
of human breast cancer cells MCF-7

| | | 72 h | |
|---|---|---|---|
| | | | Proliferation |
| Group | | OD | inhibition (%) |
| Blank well | | 0.234 ± 0.002 | — |
| Negative control group | | 1.635 ± 0.013 | — |
| Administration group | 200 μM | 0.814 ± 0.047** | 50.194 ± 2.901** |
| | 100 μM | 0.973 ± 0.027** | 40.469 ± 1.643** |
| | 50 μM | 1.281 ± 0.040** | 21.631 ± 2.470** |

Note:
Compared with the negative control group,
*P < 0.05,
**P < 0.01.

TABLE 19
Inhibitory effect of micropeptide MIAC on the proliferation
of human ovarian cancer cells SKOV3

| | | 72 h | |
|---|---|---|---|
| | | | Proliferation |
| Group | | OD | inhibition (%) |
| Blank well | | 0.205 ± 0.002 | — |
| Negative control group | | 1.674 ± 0.017 | — |
| Administration group | 200 μM | 0.727 ± 0.038** | 56.600 ± 2.243** |
| | 100 μM | 0.843 ± 0.049** | 49.652 ± 2.934** |
| | 50 μM | 1.244 ± 0.282** | 25.702 ± 16.864** |

Note:
Compared with the negative control group,
*P < 0.05,
**P < 0.01.

TABLE 20
Inhibitory effect of micropeptide MIAC on the proliferation
of human ovarian cancer cells A2780

| | | 72 h | |
|---|---|---|---|
| | | | Proliferation |
| Group | | OD | inhibition (%) |
| Blank well | | 0.215 ± 0.002 | — |
| Negative control group | | 1.195 ± 0.008 | — |
| Administration group | 200 μM | 0.441 ± 0.018** | 63.114 ± 1.519** |
| | 100 μM | 0.791 ± 0.067** | 33.789 ± 5.608** |
| | 50 μM | 0.985 ± 0.046** | 17.550 ± 3.842** |

Note:
Compared with the negative control group,
*P < 0.05,
**P < 0.01.

TABLE 21
Inhibitory effect of micropeptide MIAC on the proliferation
of human cervical cancer cells Hela

| | | 72 h | |
|---|---|---|---|
| | | | Proliferation |
| Group | | OD | inhibition (%) |
| Blank well | | 0.291 ± 0.002 | — |
| Negative control group | | 1.181 ± 0.018 | — |
| Administration group | 200 μM | 0.532 ± 0.066** | 54.941 ± 5.616** |
| | 100 μM | 0.801 ± 0.039** | 32.185 ± 3.281** |
| | 50 μM | 0.954 ± 0.041** | 19.170 ± 3.432** |

Note:
Compared with the negative control group,
*P < 0.05,
**P < 0.01.

TABLE 22
Inhibitory effect of micropeptide MIAC on the proliferation
of human cervical cancer cells SIHA

| | | 72 h | |
|---|---|---|---|
| | | | Proliferation |
| Group | | OD | inhibition (%) |
| Blank well | | 0.291 ± 0.002 | — |
| Negative control group | | 1.030 ± 0.022 | — |
| Administration group | 200 μM | 0.261 ± 0.052** | 74.693 ± 5.023** |
| | 100 μM | 0.755 ± 0.103** | 26.667 ± 10.000** |
| | 50 μM | 0.844 ± 0.099** | 18.026 ± 9.582** |

Note:
Compared with the negative control group,
*P < 0.05,
**P < 0.01.

TABLE 23
Inhibitory effect of micropeptide MIAC on the proliferation
of human non-small cell lung cancer cells NCI-H1299

| | | 72 h | |
|---|---|---|---|
| | | | Proliferation |
| Group | | OD | inhibition (%) |
| Blank well | | 0.291 ± 0.002 | — |
| Negative control group | | 1.227 ± 0.020 | — |
| Administration group | 200 μM | 0.467 ± 0.012** | 61.950 ± 1.001** |
| | 100 μM | 0.754 ± 0.06** | 38.539 ± 4.892** |
| | 50 μM | 1.090 ± 0.022 | 11.162 ± 1.793 |

Note:
Compared with the negative control group,
*P < 0.05,
**P < 0.01.

TABLE 24
Inhibitory effect of micropeptide MIAC on the proliferation
of human non-small cell lung cancer cells NCI-H1650

| | | 72 h | |
|---|---|---|---|
| | | | Proliferation |
| Group | | OD | inhibition (%) |
| Blank well | | 0.225 ± 0.002 | — |
| Negative control group | | 1.208 ± 0.016 | — |
| Administration group | 200 μM | 0.779 ± 0.057** | 35.559 ± 4.721** |
| | 100 μM | 0.955 ± 0.031** | 20.938 ± 2.565** |
| | 50 μM | 1.127 ± 0.022 | 6.703 ± 1.851 |

Note:
Compared with the negative control group,
*P < 0.05,
**P < 0.01.

TABLE 25
Inhibitory effect of micropeptide MIAC on the proliferation
of human lung giant cell carcinoma cells 95-D

| | | 72 h | |
|---|---|---|---|
| | | | Proliferation |
| Group | | OD | inhibition (%) |
| Blank well | | 0.218 ± 0.003 | — |
| Negative control group | | 1.226 ± 0.010 | — |
| Administration group | 200 μM | 0.341 ± 0.010** | 72.151 ± 0.817** |
| | 100 μM | 0.863 ± 0.054** | 29.589 ± 4.418** |
| | 50 μM | 1.188 ± 0.022 | 3.046 ± 1.818 |

Note:
Compared with the negative control group,
*P < 0.05,
**P < 0.01.

TABLE 26
Inhibitory effect of micropeptide MIAC on the proliferation
of human bladder transitional cell carcinoma cells T24

| | | 72 h | |
|---|---|---|---|
| | | | Proliferation |
| Group | | OD | inhibition (%) |
| Blank well | | 0.312 ± 0.003 | — |
| Negative control group | | 1.134 ± 0.007 | — |
| Administration group | 200 μM | 0.364 ± 0.043** | 67.901 ± 3.786** |
| | 100 μM | 0.730 ± 0.085** | 35.626 ± 7.471** |
| | 50 μM | 1.125 ± 0.010 | 0.794 ± 0.869 |

Note:
Compared with the negative control group,
*P < 0.05,
**P < 0.01.

TABLE 27
Inhibitory effect of micropeptide MIAC on the
proliferation of human bladder cancer cells EJ

| | | 72 h | |
|---|---|---|---|
| | | | Proliferation |
| Group | | OD | inhibition (%) |
| Blank well | | 0.312 ± 0.003 | — |
| Negative control group | | 1.123 ± 0.007 | — |
| Administration group | 200 μM | 0.628 ± 0.045** | 44.125 ± 3.964** |
| | 100 μM | 0.891 ± 0.009** | 20.682 ± 0.761** |
| | 50 μM | 1.126 ± 0.017 | −0.208 ± 1.490 |

Note:
Compared with the negative control group,
*P < 0.05,
**P < 0.01.

TABLE 28
Inhibitory effect of micropeptide MIAC on the proliferation
of human malignant melanoma cells A375

| | | 72 h | |
|---|---|---|---|
| | | | Proliferation |
| Group | | OD | inhibition (%) |
| Blank well | | 0.304 ± 0.003 | — |
| Negative control group | | 1.544 ± 0.027 | — |
| Administration group | 200 μM | 0.911 ± 0.071** | 41.032 ± 4.600** |
| | 100 μM | 1.194 ± 0.043** | 22.664 ± 2.800** |
| | 50 μM | 1.310 ± 0.069** | 15.174 ± 4.488** |

Note:
Compared with the negative control group,
*P < 0.05,
**P < 0.01.

TABLE 29
Inhibitory effect of micropeptide MIAC on the proliferation
of human malignant melanoma cells SK-mel-2

| | | 72 h | |
|---|---|---|---|
| | | | Proliferation |
| Group | | OD | inhibition (%) |
| Blank well | | 0.234 ± 0.002 | — |
| Negative control group | | 1.567 ± 0.015 | — |
| Administration group | 200 μM | 0.570 ± 0.011** | 63.632 ± 0.672** |
| | 100 μM | 0.822 ± 0.060** | 47.554 ± 3.816** |
| | 50 μM | 1.302 ± 0.043** | 16.908 ± 2.713** |

Note:
Compared with the negative control group,
*P < 0.05,
**P < 0.01.

TABLE 30
Inhibitory effect of micropeptide MIAC on the proliferation
of human malignant melanoma cells M14

| | | 72 h | |
|---|---|---|---|
| | | | Proliferation |
| Group | | OD | inhibition (%) |
| Blank well | | 0.234 ± 0.002 | — |
| Negative control group | | 1.690 ± 0.029 | — |
| Administration group | 200 μM | 0.639 ± 0.046** | 62.162 ± 2.695** |
| | 100 μM | 1.046 ± 0.060** | 38.075 ± 3.523** |
| | 50 μM | 1.585 ± 0.030 | 6.195 ± 1.791 |

Note:
Compared with the negative control group,
*P < 0.05,
**P < 0.01.

TABLE 31
Inhibitory effect of micropeptide MIAC on the proliferation
of human cerebral astrocytic glioblastoma cells U87-MG

| | | 72 h | |
|---|---|---|---|
| | | | Proliferation |
| Group | | OD | inhibition (%) |
| Blank well | | 0.225 ± 0.002 | — |
| Negative control group | | 1.040 ± 0.038 | — |
| Administration group | 200 μM | 0.656 ± 0.030** | 36.955 ± 2.912** |
| | 100 μM | 0.785 ± 0.029** | 24.519 ± 2.802** |
| | 50 μM | 0.951 ± 0.019 | 8.590 ± 1.860 |

Note:
Compared with the negative control group,
*P < 0.05,
**P < 0.01.

TABLE 32
Inhibitory effect of micropeptide MIAC on the proliferation
of human neuroblastoma cells SH-SY5Y

| | | 72 h | |
|---|---|---|---|
| | | | Proliferation |
| Group | | OD | inhibition (%) |
| Blank well | | 0.225 ± 0.002 | — |
| Negative control group | | 1.170 ± 0.009 | — |
| Administration group | 200 μM | 0.665 ± 0.011** | 43.175 ± 0.922** |
| | 100 μM | 0.822 ± 0.108** | 29.752 ± 9.195** |
| | 50 μM | 1.033 ± 0.022 | 11.656 ± 1.912 |

Note:
Compared with the negative control group,
*P < 0.05,
**P < 0.01.

TABLE 33
Inhibitory effect of micropeptide MIAC on the
proliferation of human glioma cells SHG-44

| | | 72 h | |
|---|---|---|---|
| | | | Proliferation |
| Group | | OD | inhibition (%) |
| Blank well | | 0.218 ± 0.003 | — |
| Negative control group | | 1.276 ± 0.014 | — |
| Administration group | 200 μM | 0.393 ± 0.021** | 69.183 ± 1.606** |
| | 100 μM | 0.838 ± 0.067** | 34.369 ± 5.261** |
| | 50 μM | 1.201 ± 0.026 | 5.876 ± 2.067 |

Note:
Compared with the negative control group,
*P < 0.05,
**P < 0.01.

TABLE 34
Inhibitory effect of micropeptide MIAC on the
proliferation of human osteosarcoma cells MG-63

| | | 72 h | |
|---|---|---|---|
| | | | Proliferation |
| Group | | OD | inhibition (%) |
| Blank well | | 0.198 ± 0.003 | — |
| Negative control group | | 1.279 ± 0.025 | — |
| Administration group | 200 μM | 0.365 ± 0.009** | 71.488 ± 0.665** |
| | 100 μM | 0.691 ± 0.033** | 45.999 ± 2.541** |
| | 50 μM | 1.049 ± 0.011** | 17.983 ± 0.824** |

Note:
Compared with the negative control group,
*P < 0.05,
**P < 0.01.

TABLE 35
Inhibitory effect of micropeptide MIAC on the proliferation
of human osteogenic sarcoma cells Saos-2

| | | 72 h | |
|---|---|---|---|
| | | | Proliferation |
| Group | | OD | inhibition (%) |
| Blank well | | 0.312 ± 0.003 | — |
| Negative control group | | 1.270 ± 0.023 | — |
| Administration group | 200 μM | 0.627 ± 0.052** | 50.669 ± 4.107** |
| | 100 μM | 0.946 ± 0.016** | 25.558 ± 1.220** |
| | 50 μM | 1.198 ± 0.067 | 5.694 ± 5.302 |

Note:
Compared with the negative control group,
*P < 0.05,
**P < 0.01.

TABLE 36
Inhibitory effect of micropeptide MIAC on the
proliferation of human osteosarcoma cells U-2OS

| | | 72 h | |
|---|---|---|---|
| | | | Proliferation |
| Group | | OD | inhibition (%) |
| Blank well | | 0.323 ± 0.002 | — |
| Negative control group | | 0.922 ± 0.007 | — |
| Administration group | 200 μM | 0.350 ± 0.013** | 61.989 ± 1.457** |
| | 100 μM | 0.596 ± 0.061** | 35.298 ± 6.605** |
| | 50 μM | 0.887 ± 0.024 | 3.761 ± 2.606 |

Note:
Compared with the negative control group,
*P < 0.05,
**P < 0.01.

TABLE 37
Inhibitory effect of micropeptide MIAC on the proliferation
of human histiocytic lymphoma cells U937

| | | 72 h | |
|---|---|---|---|
| | | | Proliferation |
| Group | | OD | inhibition (%) |
| Blank well | | 0.257 ± 0.003 | — |
| Negative control group | | 1.198 ± 0.025 | — |
| Administration group | 200 μM | 0.684 ± 0.046** | 42.877 ± 3.800** |
| | 100 μM | 0.821 ± 0.047** | 31.469 ± 3.907** |
| | 50 μM | 1.069 ± 0.028 | 10.740 ± 2.338 |

Note:
Compared with the negative control group,
*P < 0.05,
**P < 0.01.

TABLE 38
Inhibitory effect of micropeptide MIAC on the
proliferation of human lymphoma cells Raji

| | | 72 h | |
|---|---|---|---|
| | | | Proliferation |
| Group | | OD | inhibition (%) |
| Blank well | | 0.323 ± 0.002 | — |
| Negative control group | | 0.989 ± 0.016 | — |
| Administration group | 200 μM | 0.421 ± 0.029** | 57.432 ± 2.890** |
| | 100 μM | 0.687 ± 0.019** | 30.502 ± 1.951** |
| | 50 μM | 0.964 ± 0.037 | 2.494 ± 3.724 |

Note:
Compared with the negative control group,
*P < 0.05,
**P < 0.01.

TABLE 39
Inhibitory effect of micropeptide MIAC on the proliferation
of human diffuse large B-cell lymphoma cells U2932

| | | 72 h | |
|---|---|---|---|
| | | | Proliferation |
| Group | | OD | inhibition (%) |
| Blank well | | 0.183 ± 0.002 | — |
| Negative control group | | 0.833 ± 0.025 | — |
| Administration group | 200 μM | 0.167 ± 0.025** | 79.952 ± 3.030** |
| | 100 μM | 0.275 ± 0.038** | 67.027 ± 4.613** |
| | 50 μM | 0.426 ± 0.019** | 48.860 ± 2.290** |

Note:
Compared with the negative control group,
*P < 0.05,
**P < 0.01.

TABLE 40
Inhibitory effect of micropeptide MIAC on the proliferation
of human diffuse large B-cell lymphoma cells WSU-DLCL2

| | | 72 h | |
|---|---|---|---|
| | | | Proliferation |
| Group | | OD | inhibition (%) |
| Blank well | | 0.310 ± 0.007 | — |
| Negative control group | | 0.989 ± 0.005 | — |
| Administration group | 200 μM | 0.264 ± 0.056** | 73.306 ± 5.703** |
| | 100 μM | 0.684 ± 0.039** | 30.839 ± 3.947** |
| | 50 μM | 0.908 ± 0.061 | 8.156 ± 6.184 |

Note:
Compared with the negative control group,
*P < 0.05,
**P < 0.01.

TABLE 41
Inhibitory effect of micropeptide MIAC on the proliferation
of human diffuse large B-cell lymphoma cells OCI-LY10

| | | 72 h | |
|---|---|---|---|
| | | | Proliferation |
| Group | | OD | inhibition (%) |
| Blank well | | 0.192 ± 0.003 | — |
| Negative control group | | 0.826 ± 0.030 | — |
| Administration group | 200 μM | 0.376 ± 0.019** | 54.421 ± 2.289** |
| | 100 μM | 0.598 ± 0.033** | 27.574 ± 4.002** |
| | 50 μM | 0.719 ± 0.028** | 12.959 ± 3.388** |

Note:
Compared with the negative control group,
*P < 0.05,
**P < 0.01.

TABLE 42
Inhibitory effect of micropeptide MIAC on the proliferation
of human mantle cell lymphoma cells JeKo-1

| | | 72 h | |
|---|---|---|---|
| | | | Proliferation |
| Group | | OD | inhibition (%) |
| Blank well | | 0.310 ± 0.007 | — |
| Negative control group | | 1.273 ± 0.041 | — |
| Administration group | 200 μM | 0.496 ± 0.052** | 61.021 ± 4.051** |
| | 100 μM | 0.820 ± 0.078** | 35.602 ± 6.132** |
| | 50 μM | 1.174 ± 0.027 | 7.827 ± 2.109 |

Note:
Compared with the negative control group,
*P < 0.05,
**P < 0.01.

TABLE 43
Inhibitory effect of micropeptide MIAC on the proliferation
of human acute promyelocytic leukemia cells NB4

| | | 72 h | |
|---|---|---|---|
| | | | Proliferation |
| Group | | OD | inhibition (%) |
| Blank well | | 0.257 ± 0.003 | — |
| Negative control group | | 1.364 ± 0.016 | — |
| Administration group | 200 μM | 0.624 ± 0.052** | 54.217 ± 3.817** |
| | 100 μM | 0.826 ± 0.066** | 39.404 ± 4.803** |
| | 50 μM | 1.177 ± 0.042** | 13.689 ± 3.103** |

Note:
Compared with the negative control group,
*P < 0.05,
**P < 0.01.

TABLE 44
Inhibitory effect of micropeptide MIAC on the proliferation
of human acute non-T non-B lymphocytic leukemia cells Reh

| | | 72 h | |
|---|---|---|---|
| | | | Proliferation |
| Group | | OD | inhibition (%) |
| Blank well | | 0.323 ± 0.002 | — |
| Negative control group | | 0.989 ± 0.016 | — |
| Administration group | 200 μM | 0.421 ± 0.029** | 57.432 ± 2.890** |
| | 100 μM | 0.687 ± 0.019** | 30.502 ± 1.951** |
| | 50 μM | 0.964 ± 0.037 | 2.494 ± 3.724 |

Note:
Compared with the negative control group,
*P < 0.05,
**P < 0.01.

TABLE 45
Inhibitory effect of micropeptide MIAC on the proliferation
of human chronic myeloid leukemia cells K562

| | | 72 h | |
|---|---|---|---|
| | | | Proliferation |
| Group | | OD | inhibition (%) |
| Blank well | | 0.183 ± 0.002 | — |
| Negative control group | | 1.206 ± 0.004 | — |
| Administration group | 200 μM | 0.338 ± 0.025** | 71.993 ± 2.074** |
| | 100 μM | 0.544 ± 0.062** | 54.907 ± 5.168** |
| | 50 μM | 1.004 ± 0.012** | 16.699 ± 0.990** |

Note:
Compared with the negative control group,
*P < 0.05,
**P < 0.01.

TABLE 46
Inhibitory effect of micropeptide MIAC on the proliferation
of human monocytic leukemia cells Thp-1

| | | 72 h | |
|---|---|---|---|
| | | | Proliferation |
| Group | | OD | inhibition (%) |
| Blank well | | 0.310 ± 0.007 | — |
| Negative control group | | 1.335 ± 0.012 | — |
| Administration group | 200 μM | 0.681 ± 0.045** | 49.014 ± 3.359** |
| | 100 μM | 0.841 ± 0.058** | 36.979 ± 4.341** |
| | 50 μM | 1.265 ± 0.059 | 5.243 ± 4.414 |

Note:
Compared with the negative control group,
*P < 0.05,
**P < 0.01.

TABLE 47
Inhibitory effect of micropeptide MIAC on the proliferation
of human T-cell acute lymphoblastic leukemia cells JurKat

| | | 72 h | |
|---|---|---|---|
| | | | Proliferation |
| Group | | OD | inhibition (%) |
| Blank well | | 0.291 ± 0.002 | — |
| Negative control group | | 1.079 ± 0.029 | — |
| Administration group | 200 μM | 0.559 ± 0.038** | 48.146 ± 3.543** |
| | 100 μM | 0.730 ± 0.032** | 32.355 ± 2.996** |
| | 50 μM | 0.984 ± 0.032 | 8.776 ± 3.008 |

Note:
Compared with the negative control group,
*P < 0.05,
**P < 0.01.

TABLE 48
Inhibitory effect of micropeptide MIAC on the proliferation
of human myeloid leukemia cells HL60

| | | 72 h | |
|---|---|---|---|
| | | | Proliferation |
| Group | | OD | inhibition (%) |
| Blank well | | 0.215 ± 0.002 | — |
| Negative control group | | 1.691 ± 0.018 | — |
| Administration group | 200 μM | 0.549 ± 0.023** | 67.560 ± 1.336** |
| | 100 μM | 0.725 ± 0.027** | 57.115 ± 1.600** |
| | 50 μM | 1.378 ± 0.029** | 18.506 ± 1.732** |

Note:
Compared with the negative control group,
*P < 0.05,
**P < 0.01.

TABLE 49
Inhibitory effect of micropeptide MIAC on the
proliferation of human myeloma cells NCI-H929

| | | 72 h | |
|---|---|---|---|
| | | | Proliferation |
| Group | | OD | inhibition (%) |
| Blank well | | 0.183 ± 0.002 | — |
| Negative control group | | 1.089 ± 0.005 | — |
| Administration group | 200 μM | 0.263 ± 0.026** | 75.849 ± 2.402** |
| | 100 μM | 0.427 ± 0.016** | 60.759 ± 1.429** |
| | 50 μM | 0.860 ± 0.012** | 21.028 ± 1.128** |

Note:
Compared with the negative control group,
*P < 0.05,
**P < 0.01.

TABLE 50
Inhibitory effect of micropeptide MIAC on the
proliferation of human myeloma cells ARP-1

| | | 72 h | |
|---|---|---|---|
| | | | Proliferation |
| Group | | OD | inhibition (%) |
| Blank well | | 0.310 ± 0.007 | — |
| Negative control group | | 1.081 ± 0.013 | — |
| Administration group | 200 μM | 0.433 ± 0.013** | 59.914 ± 1.171** |
| | 100 μM | 0.692 ± 0.047** | 36.016 ± 4.355** |
| | 50 μM | 0.985 ± 0.008 | 8.850 ± 0.719 |

Note:
Compared with the negative control group,
*P < 0.05,
**P < 0.01.

TABLE 51
Inhibitory effect of micropeptide MIAC on the proliferation of peripheral
blood B lymphocytes RPMI-8226 in human multiple myeloma

| | | 72 h | |
|---|---|---|---|
| | | | Proliferation |
| Group | | OD | inhibition (%) |
| Blank well | | 0.205 ± 0.002 | — |
| Negative control group | | 2.054 ± 0.033 | — |
| Administration group | 200 μM | 0.522 ± 0.028** | 74.586 ± 1.366** |
| | 100 μM | 1.001 ± 0.027** | 51.282 ± 1.326** |
| | 50 μM | 2.008 ± 0.023 | 2.256 ± 1.100 |

Note:
Compared with the negative control group,
*P < 0.05,
**P < 0.01.

TABLE 52
Inhibitory effect of micropeptide MIAC on the proliferation
of human cholangiocarcinoma cells QBC939

| | | 72 h | |
|---|---|---|---|
| | | | Proliferation |
| Group | | OD | inhibition (%) |
| Blank well | | 0.218 ± 0.003 | — |
| Negative control group | | 1.134 ± 0.029 | — |
| Administration group | 200 μM | 0.468 ± 0.045** | 58.718 ± 3.932** |
| | 100 μM | 0.757 ± 0.028** | 33.255 ± 2.467** |
| | 50 μM | 1.13 ± 0.053 | 0.294 ± 4.643 |

Note:
Compared with the negative control group,
*P < 0.05,
**P < 0.01.

As shown in Tables 2-52, compared with the negative control group, the micropeptide MIAC at doses of 50-200 μM can significantly inhibit the proliferation of human pancreatic cancer, hepatocellular carcinoma, colorectal cancer, ovarian cancer, cervical cancer, bladder cancer, melanoma, glioma, neuroblastoma, osteosarcoma, myeloma, lymphoma, leukemia, and cholangiocarcinoma cells to varying degrees, demonstrating a dose-dependent relationship.

### Example 3

This example provides the inhibitory effect of the micropeptide MIAC on the migration of human tumor cells.

Human prostate cancer, cervical cancer, osteosarcoma, ovarian cancer, melanoma, and hepatocellular carcinoma cells were inoculated into transwell chambers, with 100 μL per well; for an administration group, 100 μL of a micropeptide MIAC was added to each of the chambers at a different dose; and no medicament was added to a blank group. Then, 0.6 mL of a complete medium comprising 10% FBS was added to transwell lower chambers to stimulate cell migration, followed by incubation at 37° C. with 5% CO₂ for 48 h. The culture medium in the wells was discarded, and the cells were fixed with methanol at room temperature for 30 min, stained with 0.1% crystal violet at room temperature for 10 min, and rinsed thoroughly with clear water; the unmigrated cells in the upper layer were wiped off with a cotton swab; and the migrated cells were observed under a microscope, and four visual fields were selected for photography and counting. The migration inhibition rate (MI) was calculated using the following formula:

$$MI ( \% ) = ( 1 - \frac{{N}_{test}}{{N}_{control}} ) \times 100 \%$$

where Nₜₑₛₜ represents the number of migrated cells in the administration group, and N_{control} represents the number of migrated cells in the blank control group.

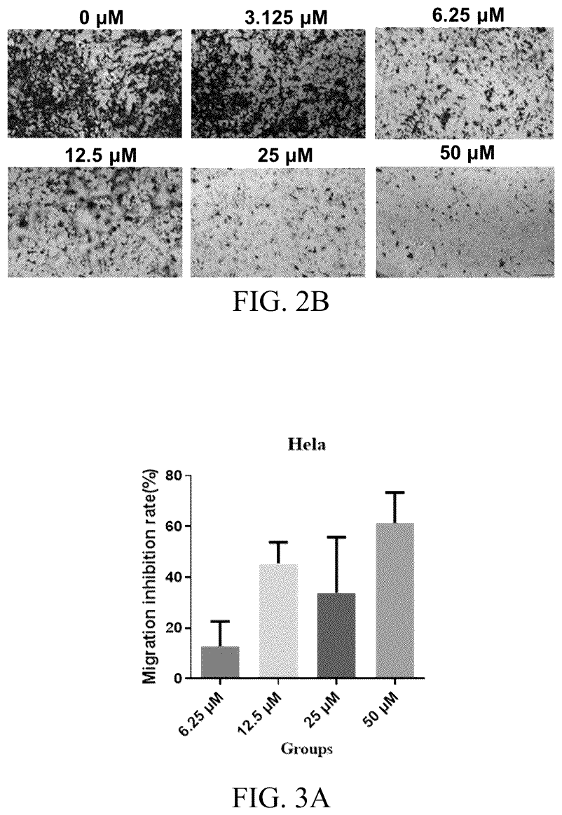

The results are shown in Tables 53-58 and FIG. 2-FIG. 7.

TABLE 53
Inhibitory effect of MIAC on the migration
activity of prostate cancer Du145 cells

| | | Dose | Number of | Inhibition |
|---|---|---|---|---|
| Group | Medicament | (μmol/Lμmol/L) | migrated cells | rate (%) |
| G1 | None | 0 | 574.8 ± 106.9 | — |
| G2 | MIAC | 3.125 | 349.3 ± 98.5 | 39.23 |
| G3 | | 6.25 | 173.8 ± 48.8 | 69.77 |
| G4 | | 12.5 | 100.5 ± 11.8 | 82.51 |
| G5 | | 25 | 46.8 ± 8.5 | 91.87 |
| G6 | | 50 | 31.3 ± 8.4 | 94.56 |

FIG. 2A corresponds to the statistical graph of all the groups in Table 53; and FIG. 2B corresponds to the staining images of cells in all the groups in Table 53 above.

TABLE 54
Inhibitory effect of MIAC on the migration
activity of cervical cancer Hela cells

| | | Dose | Number of | Inhibition |
|---|---|---|---|---|
| Group | Medicament | (μmol/L) | migrated cells | rate (%) |
| G1 | None | 0 | 544.5 ± 132.8 | — |
| G2 | MIAC | 6.25 | 475.0 ± 53.3 | 12.76 |
| G3 | | 12.5 | 296.8 ± 45.0 | 39.23 |
| G4 | | 25 | 360.3 ± 119.4 | 33.83 |
| G5 | | 50 | 210.5 ± 66.6 | 61.30 |

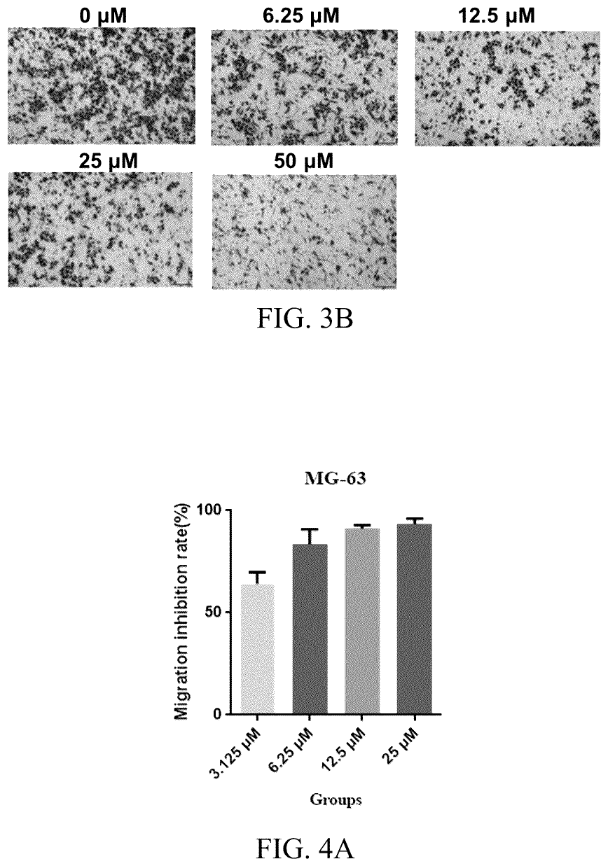

FIG. 3A corresponds to the statistical graph of all the groups in Table 54; and FIG. 3B corresponds to the staining images of cells in all the groups in Table 54 above.

TABLE 55
Inhibitory effect of MIAC on the migration
activity of osteosarcoma MG-63 cells

| | | Dose | Number of | Inhibition |
|---|---|---|---|---|
| Group | Medicament | (μmol/L) | migrated cells | rate (%) |
| G1 | None | 0 | 360.3 ± 123.0 | — |
| G2 | MIAC | 3.125 | 129.5 ± 19.9 | 64.05 |
| G3 | | 6.25 | 60.5 ± 26.7 | 83.21 |
| G4 | | 12.5 | 32.5 ± 6.0 | 90.98 |
| G5 | | 25 | 24.8 ± 9.6 | 93.13 |

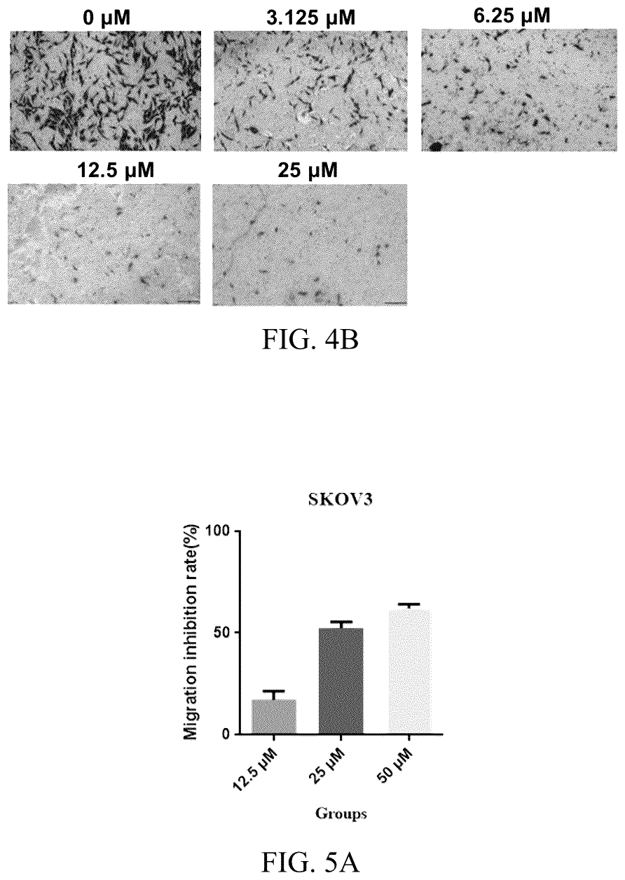

FIG. 4A corresponds to the statistical graph of all the groups in Table 55; and FIG. 4B corresponds to the staining images of cells in all the groups in Table 55 above.

TABLE 56
Inhibitory effect of MIAC on the migration
activity of ovarian cancer SKOV3 cells

| | | Dose | Number of | Inhibition |
|---|---|---|---|---|
| Group | Medicament | (μmol/L) | migrated cells | rate (%) |
| G1 | None | 0 | 1095.3 ± 63.9 | — |
| G2 | MIAC | 12.5 | 907.3 ± 46.5 | 17.17 |
| G3 | | 25 | 523.8 ± 35.3 | 52.28 |
| G4 | | 50 | 415.8 ± 21.7 | 62.04 |

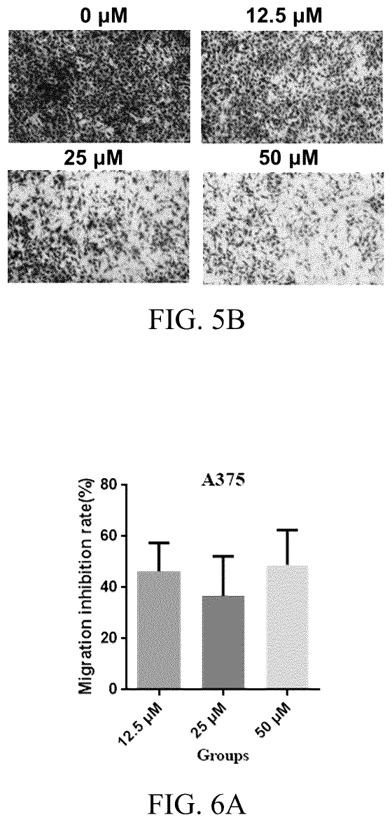

FIG. 5A corresponds to the statistical graph of all the groups in Table 56; and FIG. 5B corresponds to the staining images of cells in all the groups in Table 56 above.

TABLE 57
Inhibitory effect of MIAC on the migration
activity of malignant melanoma A375 cells

| | | Dose | Number of | Inhibition |
|---|---|---|---|---|
| Group | Medicament | (μmol/L) | migrated cells | rate (%) |
| G1 | None | 0 | 258.8 ± 26.3 | — |
| G2 | MIAC | 12.5 | 139.3 ± 28.5 | 46.18 |
| G3 | | 25 | 164.3 ± 40.1 | 36.52 |
| G4 | | 50 | 132.8 ± 35.1 | 48.70 |

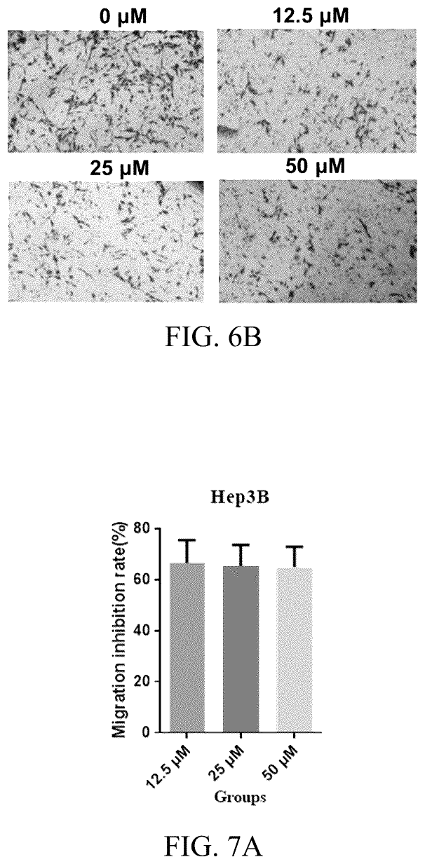

FIG. 6A corresponds to the statistical graph of all the groups in Table 57; and FIG. 6B corresponds to the staining images of cells in all the groups in Table 57 above.

TABLE 58
Inhibitory effect of MIAC on the migration activity
of hepatocellular carcinoma Hep3B cells

| | | Dose | Number of | Inhibition |
|---|---|---|---|---|
| Group | Medicament | (μmol/L) | migrated cells | rate (%) |
| G1 | None | 0 | 331.0 ± 18.2 | — |
| G2 | MIAC | 12.5 | 110.8 ± 29.3 | 66.54 |
| G3 | | 25 | 114.8 ± 27.1 | 65.33 |
| G4 | | 50 | 116.0 ± 26.1 | 64.95 |

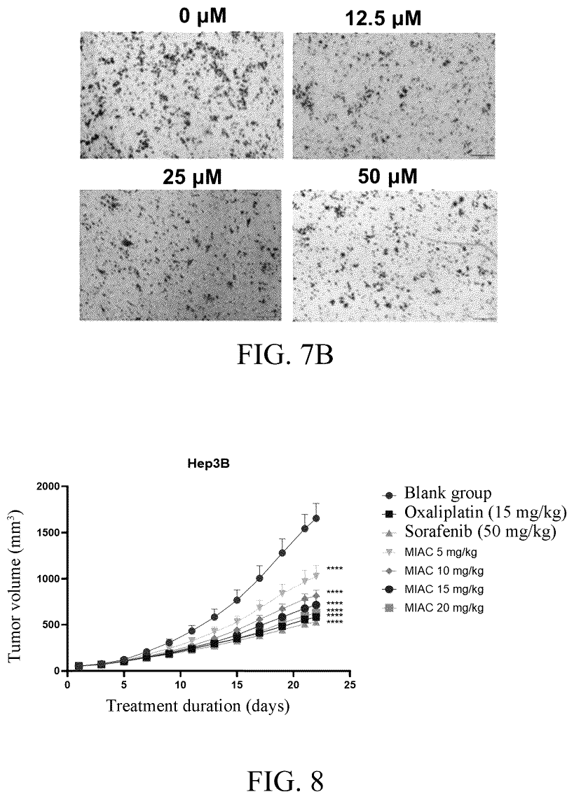

FIG. 7A corresponds to the statistical graph of all the groups in Table 58; and FIG. 7B corresponds to the staining images of cells in all the groups in Table 58 above.

It can be seen from Tables 53-58 and FIG. 2-FIG. 7 that, compared with the blank control group, the micropeptide MIAC has a significant inhibitory effect on the migration of human prostate cancer Du145 cells, human osteosarcoma MG-63 cells, human ovarian cancer SKOV3 cells, human hepatocellular carcinoma Hep3B cells, human melanoma A375 cells, and human cervical cancer Hela cells within a certain range, and the inhibition rate is dose-dependent, indicating that the micropeptide MIAC can be developed into anti-tumor medicaments by inhibiting the migration of the above malignant tumor cells.

### Example 4

This example provides the inhibitory effect of a micropeptide MIAC on the in vivo growth of human tumor cells.

Human hepatocellular carcinoma Hep3B cells were cultured in large quantities and digested with a 0.25% trypsin solution; and the cell suspension after digestion termination was centrifuged at 1000 rpm for 5 minutes, and the cells were resuspended in a serum-free MEM medium and then counted to adjust the cell concentration to 5×10⁷ cells/mL. Each BALB/c nude mouse (4-6 weeks old, female, weighing 14-16 g, adaptively housed in an SPF animal breeding room for 1 week) was inoculated with 100 μL of the cell suspension in the left axilla, with an inoculated cell amount of 5×10⁶ cells. After inoculation, the tumor growth at the inoculation site of nude mice was closely observed. On the 7^{th} day after inoculation, white nodules appeared at the inoculation site, which could be moved subcutaneously upon touch. As the tumor tissue grew, a hard tumor mass gradually formed at the inoculation site. Approximately 14 days later, the average volume of the tumor tissue reached 100 mm³, the BALB/c nude mice were randomly divided into 7 groups (a normal saline group was blank control, the dosage of the micropeptide MIAC at 5 mg/kg was considered as a low dose, the dosage of the micropeptide MIAC at 10 mg/kg was considered as a medium dose, the dosage of the micropeptide MIAC at 15 mg/kg was considered as a medium-high dose, the dosage of the micropeptide MIAC at 20 mg/kg was considered as a high dose, and 15 mg/kg oxaliplatin and 50 mg/kg sorafenib were positive controls), and there were 10 mice in the blank group and 6 mice in each of the other groups, with the body weight of the mice being about 20 g at the start of administration. The volume of the transplanted tumor was measured and recorded every two days, and the formula for calculating the tumor volume (TV) was: tumor volume=0.5×a×b², where a is the length (mm) of the transplanted tumor and b is the width (mm) of the transplanted tumor.

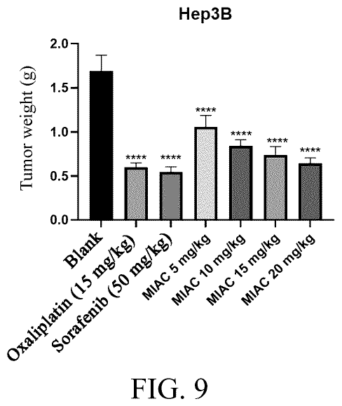

The results are shown in FIG. 8 and FIG. 9. Compared with the blank control group, the micropeptide MIAC can significantly inhibit the in vivo tumor growth of human hepatocellular carcinoma Hep3B cells to varying degrees, demonstrating a dose-dependent relationship. At a dose of 20 mg/kg, its therapeutic effect on tumors is similar to that of positive medicaments, indicating that the micropeptide MIAC has a good anti-tumor effect in vivo.

What is claimed is:

1. Use of a micropeptide MIAC in preparation of a reagent or a medicament for detecting, preventing or treating tumors, wherein the micropeptide MIAC has an amino acid sequence as shown in SEQ ID NO: 1; and the tumors comprise one or more of pancreatic cancer, hepatocellular carcinoma, colorectal cancer, ovarian cancer, cervical cancer, bladder cancer, melanoma, glioblastoma, neuroblastoma, glioma, osteosarcoma, lymphoma, hematologic malignancies, myeloma, cholangiocarcinoma and prostate cancer.

2. The use of a micropeptide MIAC in preparation of a reagent or a medicament for detecting, preventing or treating tumors according to claim 1, wherein the reagent or the medicament for preventing or treating tumors comprises at least the micropeptide MIAC and a pharmaceutically acceptable carrier thereof.

3. The use of a micropeptide MIAC in preparation of a reagent or a medicament for detecting, preventing or treating tumors according to claim 1, wherein the preventing or treating tumors comprises inhibiting growth, migration and/or proliferation of the tumor cells.

4. The use of a micropeptide MIAC in preparation of a reagent or a medicament for detecting, preventing or treating tumors according to claim 3, wherein the preventing or treating tumors comprises inhibiting the proliferation of the tumor cells,
cancer cells of the pancreatic cancer comprise any one or a combination of MIP-PaCa-2, Aspc-1 and PANC-1;
cancer cells of the hepatocellular carcinoma comprise any one or a combination of HepG2, SMMC-7721, HCCLM3, QGY-7701, and Hep3B;
cancer cells of the colorectal cancer comprise any one or a combination of LOVO, SW480, and HCT-116;
cancer cells of the ovarian cancer comprise SKOV3 and/or A2780;
cancer cells of the cervical cancer comprise Hela and/or SIHA;
cancer cells of the bladder cancer comprise T24 and/or EJ;
cancer cells of the melanoma comprise any one or a combination of A375, SK-mel-2, and M14;
cancer cells of the glioblastoma comprise U87-MG;
cancer cells of the neuroblastoma comprise SH-SY5Y;
cancer cells of the glioma comprise SHG-44;
cancer cells of the osteosarcoma comprise any one or a combination of MG-63, Saos-2, and U-2OS;
cancer cells of the lymphoma comprise any one or a combination of U937, Raji, U2932, WSU-DLCL2, OCI-LY10, and JeKo-1;
cancer cells of the hematologic malignancies comprise any one or a combination of NB4, Reh, K562, Thp-1, JurKat, and HL60;
cancer cells of the myeloma comprise any one or a combination of NCI-H929, ARP-1 and RPMI-8226;
cancer cells of the cholangiocarcinoma comprise QBC939;
and/or,
cancer cells of the prostate cancer comprise Du145 cells.

5. The use of a micropeptide MIAC in preparation of a reagent or a medicament for detecting, preventing or treating tumors according to claim 3, wherein the preventing or treating tumors comprises inhibiting the migration of prostate cancer Du145 cells, cervical cancer Hela cells, osteosarcoma MG-63 cells, ovarian cancer SKOV3 cells, melanoma A375 cells, and/or hepatocellular carcinoma Hep3B cells.

6. The use of a micropeptide MIAC in preparation of a reagent or a medicament for detecting, preventing or treating tumors according to claim 3, wherein the preventing or treating tumors comprises inhibiting the growth of hepatocellular carcinoma Hep3B cells.

7. Use of a nucleotide in preparation of a reagent or a medicament for detecting, preventing or treating tumors, wherein the nucleotide encodes an amino acid sequence as shown in SEQ ID NO: 1; and the tumors comprise one or more of pancreatic cancer, hepatocellular carcinoma, colorectal cancer, ovarian cancer, cervical cancer, bladder cancer, melanoma, glioblastoma, neuroblastoma, glioma, osteosarcoma, lymphoma, hematologic malignancies, myeloma, cholangiocarcinoma and prostate cancer.

8. The use of nucleotide in preparation of a reagent or a medicament for detecting, preventing or treating tumors according to claim 7, wherein the nucleotide comprises a nucleotide sequence as shown in SEQ ID NO: 2.

9. Use of a recombinant vector in preparation of a reagent or a medicament for detecting, preventing or treating tumors, wherein the recombinant vector encodes an amino acid sequence as shown in SEQ ID NO: 1 or comprises a nucleotide sequence as shown in SEQ ID NO: 2; and the tumors comprise one or more of pancreatic cancer, hepatocellular carcinoma, colorectal cancer, ovarian cancer, cervical cancer, bladder cancer, melanoma, glioblastoma, neuroblastoma, glioma, osteosarcoma, lymphoma, hematologic malignancies, myeloma, cholangiocarcinoma and prostate cancer.

10. A pharmaceutical composition for treating tumors, wherein the pharmaceutical composition comprises at least the micropeptide MIAC according to claim 1, and a pharmaceutically acceptable carrier thereof; and the tumors comprise one or more of pancreatic cancer, hepatocellular carcinoma, colorectal cancer, ovarian cancer, cervical cancer, bladder cancer, melanoma, glioblastoma, neuroblastoma, glioma, osteosarcoma, lymphoma, hematologic malignancies, myeloma, cholangiocarcinoma and prostate cancer.

11. A pharmaceutical composition for treating tumors, wherein the pharmaceutical composition comprises at least the nucleotide according to claim 7, and a pharmaceutically acceptable carrier thereof; and the tumors comprise one or more of pancreatic cancer, hepatocellular carcinoma, colorectal cancer, ovarian cancer, cervical cancer, bladder cancer, melanoma, glioblastoma, neuroblastoma, glioma, osteosarcoma, lymphoma, hematologic malignancies, myeloma, cholangiocarcinoma and prostate cancer.

12. The use of a micropeptide MIAC in preparation of a reagent or a medicament for detecting, preventing or treating tumors according to claim 2, wherein the preventing or treating tumors comprises inhibiting growth, migration and/or proliferation of the tumor cells.


SEQUENCE LISTING (4 sequences)

SEQ ID NO: 1 - protein, 51 aa, Homo sapiens
MERAGVPGFSPRRSSVEAKMQSTSCSVRKSSTVTAWPAVVLLLSWGQRRGG

SEQ ID NO: 2 - DNA, 156 bp, synthetic construct
  source 1..156: mol_type = other DNA
atggagagagcaggggtgcccgggttctctccgcggcgctcatcggtggaggcgaagatgcagagcaccagctgcagtgtcaggaagagctccacagtcaccgcctggccagccgtcgtgctgttgctgagctggggacagagaagaggcggatga

SEQ ID NO: 3 - DNA, 22 bp, synthetic construct
  source 1..22: mol_type = other DNA
atggagagagcaggggtgcccg

SEQ ID NO: 4 - DNA, 23 bp, synthetic construct
  source 1..23: mol_type = other DNA
cgcctcttctctgtccccagctc